(19) United States
(12) Patent Application Publication
(10) Pub. No.: US 2014/0187509 A1
(43) Pub. Date: Jul. 3, 2014

(54) USE OF S-ADENOSYLMETHIONINE, VITAMIN E, AND VITAMIN C FOR THE PREVENTION AND TREATMENT OF CARDIOVASCULAR DYSFUNCTION

(71) Applicants: Wilfred Wayne Lautt, Gibsons (CA); Zhi Ming, Saskatoon (CA)

(72) Inventors: Wilfred Wayne Lautt, Gibsons (CA); Zhi Ming, Saskatoon (CA)

(73) Assignee: SCIMAR LTD., Gibsons, BC (CA)

(21) Appl. No.: 14/111,882

(22) PCT Filed: Apr. 13, 2012
(86) PCT No.: PCT/CA12/50238

§ 371 Date: Feb. 27, 2014

Related U.S. Application Data
(60) Provisional application No. 61/476,014, filed on Apr. 15, 2011.

(51) Int. Cl. A61K 31/7076; A61K 31/355; A61K 31/375; A61K 31/197

(52) U.S. Cl. CPC A61K 31/7076; A61K 31/197; A61K 31/355; A61K 31/375
USPC 514/46

(57) ABSTRACT

The present invention provides a method of preventing or treating cardiovascular dysfunction by administering a therapeutically effective amount of (a) one or more of (i) S-adenosylmethionine or a derivative or pharmaceutically acceptable salt thereof and (ii) N-acetylcysteine or a derivative or pharmaceutically acceptable salt thereof; (b) vitamin E or a derivative or pharmaceutically acceptable salt thereof; and (c) vitamin C or a derivative or pharmaceutically acceptable salt thereof and a pharmaceutically acceptable carrier.

### CROSS-REFERENCE TO RELATED APPLICATION

This application claims the benefit of and priority to U.S. Patent Application No. 61/476,014 filed 15 Apr. 2011 under the title USE OF S-ADENOSYLMETHIONINE, VITAMIN E, AND VITAMIN C FOR THE PREVENTION AND TREATMENT OF CARDIOVASCULAR DYSFUNCTION. The content of the above patent application is hereby expressly incorporated by reference into the detailed description hereof.

### FIELD OF INVENTION

The present invention relates to pharmaceutical compositions, particularly those used in the prevention and treatment of cardiovascular dysfunction.

### BACKGROUND

Cardiovascular dysfunction are dysfunctions that involve the circulatory system including the heart and blood vessels that affect the lungs, the brain, kidneys or other parts of the body. Cardiovascular dysfunction can be measured by changes to the pressure-volume indexes or hemodynamic parameters, such as: preload recruitable stroke work (PRSW); end-systolic pressure-volume relationship (ESPVR); dP/dtₘₐₓ−end-diastolic volume relationship (dP/dtₘₐₓ−EDV); maximum elastance (Eₘₐₓ); maximal left ventricular systolic pressure (Pes); left ventricular end-diastolic pressure (Ped); the maximal rates of left-ventricular pressure upstroke and fall (dP/dtₘₐₓ and dP/dtₘᵢₙ, respectively); time constant of left ventricular pressure decay (tau); ejection fraction (EF); cardiac output normalized to body weight (cardiac index, CI); stroke work normalized to body weight (SWI); and, total peripheral resistance index (TPRI).

Traditional treatment and prevention of cardiovascular dysfunction involves modifications to lifestyle and diet and surgery. Medications used to prevent cardiovascular dysfunction include aspirin, digitalis, angiotensin converting enzyme (ACE) inhibitors, beta blockers, nitrates, calcium channel blockers, diuretics, blood cholesterol lowering agents, and thrombolytic agents.

A pharmaceutical composition comprising: (a) S-Adenosylmethionine, a derivative or pharmaceutically acceptable salt thereof, (b) vitamin E, a derivative or pharmaceutically acceptable salt thereof, and (c) vitamin C, a derivative or pharmaceutically acceptable salt thereof and (d) a pharmaceutically acceptable carrier (collectively, SAMEC) is known to act in synergy in the treatment of oxidative liver injury and in the treatment of Hiss Dependent Insulin Resistance (HDIR) (U.S. patent application Ser. No. 814886), and also in the prevention of HDIR that develops with aging, and the metabolic dysfunction associated with aging combined with sucrose supplementation (Lautt et al, 2008; Ming Z, et al. Can. J. Physiol. Pharmacol. 87: 873-882 (2009)). SAMEC was originally concocted by the inventor to provide antioxidant protection against the severe acute free radical hepatotoxicity generated by thioacetamide by protecting the aqueous and lipid components of the cell and the mitochondrial and glutathione levels (Lautt et al., 2008, Ming et al., 2006, Ming et al., 2009). Other roles that SAMEC may confer are unknown.

### SUMMARY OF INVENTION

The present invention provides the surprising use of the combination of therapeutically effective amounts of S-Adenosylmethionine or N-Acetylcysteine, Vitamin E, and Vitamin C for the prevention or the treatment of cardiac dysfunction.

In a first aspect, the invention provides the use of a pharmaceutical composition comprising: S-adenosylmethionine or N-Acetylcysteine, vitamin E, vitamin C and a pharmaceutically acceptable carrier for the prevention or the treatment of cardiovascular dysfunction.

Other aspects and features of the present application will become apparent to those skilled in the art upon review of the following description of specific embodiments of the application in conjunction with the accompanying figures.

### BRIEF DESCRIPTION OF DRAWINGS

In FIGS. 1 to 12, described below: “Young” represents 9-week old rats; “52C” represents 52-week old rats with a control diet; “52A” represents 52-week old rats with a SAMEC-supplemented diet; “52S” represents 52-week old rats with a diet high in sucrose; and “52T” represents 52-week old rats with a diet high in sucrose and supplemented by SAMEC.

FIG. 1 is a bar graph showing the effect of SAMEC on the pressure-volume index, end-systolic pressure-volume relationship (ESPVR), in aging rats.

FIG. 2 is a bar graph showing the effect of SAMEC on the pressure-volume index, Eₘₐₓ, in aging rats.

FIG. 3 is a bar graph showing the effect of SAMEC on the pressure-volume index, dP/dt−EDV, in aging rats.

FIG. 4 is a bar graph showing the effect of SAMEC on the pressure-volume index, preload recruitable stroke work (PRSW), in aging rats.

FIG. 5 is a bar graph showing the effect of SAMEC on the baseline hemodynamic parameter, cardiac index (CI), in aging rats.

FIG. 6 is a bar graph showing the effect of SAMEC on the baseline hemodynamic parameter, stroke work index (SWI), in aging rats.

FIG. 7 is a bar graph showing the effect of SAMEC on the baseline hemodynamic parameter, dP/dtₘₐₓ, in aging rats.

FIG. 8 is a bar graph showing the effect of SAMEC on the baseline hemodynamic parameter, −dP/dtₘᵢₙ, in aging rats.

FIG. 9 is a bar graph showing the effect of SAMEC on the baseline hemodynamic parameter, ejection fraction (EF %), in aging rats.

FIG. 10 is a bar graph showing the effect of SAMEC on the baseline hemodynamic parameter, time constant of left ventricular pressure decay (tau), in aging rats.

FIG. 11 is a bar graph showing the effect of SAMEC on the baseline hemodynamic parameter, left ventricular end-diastolic pressure (Ped), in aging rats.

FIG. 12 is a bar graph showing the effect of SAMEC on the baseline hemodynamic parameter, total peripheral resistance index (TPRI), in aging rats.

FIG. 13 is a bar graph showing the percent protection conferred by SAMEC for age-associated cardiovascular impairment, in ejection fraction, preload recruitable stroke work, maximum rate of decreased diastolic pressure, maximum rate of increased systolic pressure, dP/dtₘₐₓ−EDV, total peripheral resistance, time constant of left-ventrical pressure decay, end-systolic pressure-volume relationship, stroke work index and cardiac index.

FIG. 14 is a bar graph showing the percent protection conferred by SAMEC for age-associated and sucrose-associated cardiovascular dysfunction, in preload recruitable stroke work, maximum rate of decreased diastolic pressure, maximum rate of increased systolic pressure, end-systolic pressure-volume relationship, dP/dtₘₐₓ−EDV, ejection fraction, time constant of left-ventrical pressure decay, total peripheral resistance, stroke work index and cardiac index.

### DETAILED DESCRIPTION

The present inventors have determined that combined treatment of S-adenosylmethionine (SAMe), vitamin E and vitamin C (SAMEC) protects against cardiovascular dysfunction associated with aging, both with normal patients and patients having a diet high in sugar. S-adenosylmethionine may be substituted with N-acetylcysteine.

Aging increases oxidative stress. The present inventors concocted a unique synergistic antioxidant cocktail, abbreviated as SAMEC, consisting of S-adenosylmethionine plus vitamin E and vitamin C, to simultaneously protect the aqueous and lipid components of the cell and the mitochondrial function and glutathione levels. The cocktail was developed as a tool to protect against the severe acute free radical hepatotoxicity generated by thioacetamide. The cocktail turned out to show dramatic synergism, working only if all three components were used. The present inventors have demonstrated that impaired cardiac performance associated with aging and sucrose was protected by SAMEC.

As used herein, the term “S-adenosylmethionine” includes derivatives, conjugates and metabolites of S-adenosylmethionine and pharmaceutically acceptable salts thereof (see for example, U.S. Pat. Nos. 3,893,999 and 4,057,686). S-adenosylmethionine and its salt forms may be natural, semisynthetic, bioengineered, synthetic or extracted, or any combination thereof.

As used herein, the term “N-acetylcysteine” includes derivatives, conjugates and metabolites of S-adenosylmethionine and pharmaceutically acceptable salts thereof. N-acetylcysteine and its salt forms may be natural, semisynthetic, bioengineered, synthetic or extracted, or any combination thereof.

As used herein, the term “vitamin E” includes alpha, beta, gamma, and delta-tocopherols and their derivatives, conjugates, metabolites and salts. The vitamin E may also be a combination of alpha, beta, gamma, and delta-tocopherols. The alpha-form occurs naturally as the d-isomer known as d-.alpha.-tocopherol (d-2,5,7,8-tetramethyl-2-(4′,8′,12′-trimethyltridecyl)-6-chromanol). Other forms of vitamin E which can be used include: d-.alpha.-tocopheryl acetate, d-.alpha.-tocopheryl succinate, d-.alpha.-tocopheryl nicotinate and d-.alpha.-tocopheryl linoleate. Also the corresponding dl forms may be used which include: dl-.alpha.-tocopherol, dl-.alpha.-tocopheryl acetate, dl-.alpha.-tocopheryl succinate, dl-.alpha.-tocopheryl nicotinate and dl-.alpha.-tocopheryl linoleate and their derivatives, conjugates, metabolites and salts.

As used herein, the term “vitamin C” includes ascorbic acid and its derivatives, conjugates, metabolites and salts. Such derivatives include, for example, oxidation products such as dehydroascorbic acid and edible salts of ascorbic acid such as, illustratively, calcium, sodium, magnesium, potassium and zinc ascorbates. The term vitamin C includes these derivatives and any other art-recognized vitamin C derivatives (see for example, U.S. Pat. Nos. 5,137,723 and 5,078,989) including vitamin C esters and salts, useful for the purposes of this invention.

The S-adenosylmethionine, vitamin E, or vitamin C may be in the form of a suitable pharmaceutically acceptable salt. Functional derivatives, conjugates and metabolites of S-adenosylmethionine, vitamin E, or vitamin C can also be used to prepare the pharmaceutical composition according to the invention.

S-adenosylmethionine was selected in part because it protects hepatic mitochondria, most likely through increased production of glutathione. N-acetylcysteine does the same thing and thus N-acetylcysteine+vitamin E+vitamin C will be as effective.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The pushfit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients.

Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Suitable routes of administration may, for example, include oral delivery. Preferably, daily long term oral use is recommended for preventative use.

The methods of treating cardiovascular dysfunction according to the invention comprise the administration of a therapeutically effective amount of S-adenosylmethionine, vitamin E, vitamin C to a patient in need thereof.

By an “effective amount” or a “therapeutically effective amount” of a pharmacologically active agent is meant a nontoxic but sufficient amount of the drug or agent to provide the desired effect. In a combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. An appropriate “effective” amount in any individual case may be determined by one of ordinary skill in the art using routine experimentation.

A dosage regimen utilizing SAMEC is selected in accordance with weight of the patient and in accordance the degree of cardiovascular dysfunction clinically required to best treat the specific condition in the individual patient.

The therapeutic effective amount of any of the active agents encompassed by the invention will depend on number of factors which will be apparent to those skilled in the art and in light of the disclosure herein. In particular these factors include: the identity of the compounds to be administered, the formulation, the route of administration employed, the patient's gender, age, and weight, and the severity of the condition being treated and the presence of concurrent illness affecting the gastrointestinal tract, the hepatobillary system and the renal system. Methods for determining dosage and toxicity are well known in the art with studies generally beginning in animals and then in humans if no significant animal toxicity is observed. The patient should be monitored for signs of adverse drug reactions and toxicity, especially with regard to liver function.

For administration to mammals, and particularly humans, if administered orally, it is expected that the daily dosage level will be: from 100 mg to 900 mg of vitamin E; from 200 mg to 2000 mg of vitamin C; and, from 200 mg to 1600 mg S-adenosylmethionine, or from 400 mg to 800 mg of N-acetylcysteine. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

In a preferred embodiment of the invention, the therapeutically effective amount of the: S-adenosylmethionine is 400 mg, vitamin C is 500 mg, and vitamin E is 300 mg. S-adenosylmethionine may be substituted with N-acetylcysteine, with a preferred dose of 500 mg.

The therapeutically effective amounts may be administered in various combinations in which the components may be present in a single dosage unit or in more than one dosage unit. For example, the combinations of the present invention may be administered in a single daily dosage unit in which all components are present, e.g., in a single capsule or tablet. The doses may also be administered in combinations of more than one dosage unit in which each dosage unit contains at least one component or in which two or more components are combined into a single dosage unit. For example, a combination of S-adenosylmethionine, vitamin E and vitamin C may be administered as a pill, capsule or tablet of S-adenosylmethionine and a separate pill, tablet or capsule of vitamin E and C. A combination of S-adenosylmethionine, vitamin E and vitamin C may include each component in a separate dosage unit, or two of the components in one dosage unit, such as combined in the same capsule and the other component in a separate dosage unit, or, as explained above, all three of the components in the same (i.e., a single) dosage unit. These combinations may be provided in kits or blister packs, in which more than one dosage unit of the various components are provided in the same package or container, for co-administration to a human or animal. For example, a single dosage unit (such as for example, a tablet, a capsule) of each of S-adenosylmethionine, vitamin E and vitamin C may be placed in the same blister pack along with instructions for co-administration. These combinations may be provided, for example, in kits, blister packs, packets or bottles shrink-wrapped together in which more than one dosage unit of the various components are provided in the same dispensing unit for co-administration to a human or animal.

Although the invention has been described with reference to illustrative embodiments, it is to be understood that the invention is not limited to these precise embodiments, and that various changes and modifications may be effected therein by one skilled in the art. All such changes and modifications are intended to be encompassed in the appended claims.

### Example 1

### S-Adenosylmethionine, Vitamin E and Vitamin C in the Treatment And Protection of Cardiovascular Dysfunction

Animals and Groups: Male Sprague-Dawley rats (Charles River, St. Constant, Quebec, Canada) 7 weeks old (body weight 200-225 g) were pair housed and maintained under controlled conditions (22±1° C., 12 h light/12 h dark cycle). They were fed a standard rat chow diet (66% carbohydrates in corn starch, 20% protein and 5% lipids) with free access to water for two weeks to adapt to the housing environment. Then the animals were divided into four groups: group one (aging control) fed with normal chow; group two (SAMEC treatment) fed with normal chow supplemented with SAMEC (S-adenosylmethionine (SAMe) (0.5 g·kg diet), vitamin C (12.5 g·kg diet) and vitamin E (1.5 g·kg diet)); group 3 (sucrose) fed with normal chow with drinking water containing 5% sucrose (50 ml/rat/day plus access to regular tap water); and group four (sucrose+SAMEC treatment) fed with normal chow supplemented with SAMEC plus drinking 5% sucrose water. Given the average daily food consumption of 20 g, the approximate daily intake for vitamin C was 250 mg·kg body weight, for vitamin E 30 mg·kg body weight, and for S-adenosylmethionine 19 mg·kg body weight.

Rats included in these four groups were tested at the ages of 6 and 12 months (n=13/group). Young adult rats (n=14) at the age of 9 weeks, fed with standard rat chow, served as the young control group for 6 and 12 month old rats. Body weight gain was monitored once every two weeks. Food and water intake were monitored for 1 week periods at prescheduled times throughout the treatment. The animal identification was assured by microchip implantation.

Body weight gain was monitored once every two weeks. Food and water intake were monitored for 1 week periods at prescheduled times throughout the treatment. The animal identification was assured by microchip implantation.

Surgical Preparation: To establish a consistent postprandial state, all rats underwent an 8 hour fast and a refeeding period of 2 hours immediately before the start of surgical preparation. The rats were anesthetized with an intraperitoneal injection of sodium pentobarbital (54.7 mg/kg; CEVA Sante Animal S.A., Libourne, France). Anesthesia was maintained by a continuous infusion of pentobarbital sodium (0.5 mg/ml-1 saline given at 50 μl·min-1) through a cannula in the jugular vein, supplemented with a 0.54 mg (0.01 ml) bolus injection when required. The rats were placed on a temperature-controlled surgical table (Harvard Apparatus, Kent, England) and rectal temperature was monitored and held at 37.0-37.5° C. Spontaneous respiration was allowed through a tracheal catheter.

An arterial-venous shunt was established, as previously described (Lautt, 2003), for monitoring mean arterial blood pressure (MAP), for derivation of arterial blood samples, and for intravenous drug delivery. Briefly, two catheters (polyethylene tubing PE60), one inserted into the right femoral artery and the other into the right femoral vein, were connected with silicon tubing.

A side branch of the circuit was connected to a pressure transducer for the recording of the shunt pressure which, when the silicon tubing toward the venous side of the circuit was clamped, measured the systemic arterial blood pressure. Blood samples were taken from the arterial side of the shunt for glucose measurement. Flowing blood within the shunt assured the real time measurement of the arterial blood glucose concentration, which was essential for the dynamic euglycemic clamp test as mentioned below. An infusion line was inserted into the venous side of the shunt for intravenous drug delivery. Another infusion line connected to the jugular vein was established for glucose infusion. Animals were heparinized (100 IU.kg-1) to prevent clotting in the vascular shunt.

Hemodynamic and left ventricle pressure-volume measurement: For the assessment of hemodynamics and left ventricle (LV) pressure-volume (P-V) relationship, a microtip conductance pressure-volume (P-V) catheter (size 1.9 F, Scisense Inc., London, ON, Canada) was introduced into the right carotid artery and further advanced into the LV. The position of the catheter was carefully adjusted until stable P-V loops were obtained. The catheter was connected to an EMKA signal processor, and all data were acquired digitally and analyzed at a sample rate of 1000 Hz using IOX data acquisition/analysis system (EMKA Technologies, Falls Church, Va., USA). The abdomen was opened, and the inferior vena cava between liver and diaphragm was identified for the preparation of transient venous occlusion to decrease the cardiac preload.

The following parameters were recorded and analyzed: Heart rate (HR), mean arterial pressure (MAP), maximal left ventricular systolic pressure (Pes), left ventricular end-diastolic pressure (Ped), the maximal rates of LV pressure upstroke and fall (dP/dtmax and dP/dtmin, respectively), time constant of left ventricular pressure decay (tau), ejection fraction (EF), stroke volume (SV), cardiac output (CO), and stroke work (SW). Cardiac output was normalized to body weight (cardiac index, CI). Stroke work was also normalized to body weight (SWI). Total peripheral resistance index (TPRI) was calculated by the equation: TPRI=MAP/CI.

In addition, left ventricular pressure-volume relations were evaluated from pressure-volume loops recorded during transient occlusion of the inferior vena cava by external compression of the vessel. Preload recruitable stroke work (PRSW), end-systolic pressure-volume relationship (ESPVR), end-diastolic pressure-volume relationship (EDPVR), dP/dtmax−end-diastolic volume relationship (dP/dtmax−EDV) and maximum elastance (Emax) were calculated using IOX software.

Calibration of the conductance catheter: The volume signal of the conductance catheter was calibrated for parallel conductance and cardiac output, according to IOX recommendation. Unlike traditional indices such as ejection fraction and dP/dtmax, these additional parameters, derived from the pressure-volume relationship, are more specific and more direct indicators of ventricular performance, independent of cardiac loading conditions and heart rate (14,28). Briefly, 20 μl of 10% pre-warmed saline was injected intravenously, and, from the shift of P-V relations, parallel conductance volume was calculated by the IOX software and used for correction of the cardiac mass volume. At the end of experiments, an ultrasonic perivascular V type flow probe (size 3 mm) was placed around the arch of the thoracic aorta to measure cardiac output (T206, Transonic Systems Inc., NY, USA), for the purpose of calibrating the cardiac output measured by the conductance catheter. The heart was harvested. Left and right ventricle were separated and weighed.

Chemicals: Human Insulin was Purchased from Novo Nordisk (Bagsvaerd, Denmark). Atropine, Vitamin C (L-Ascorbic acid), and Vitamin E ((±)-α-Tocopherol) were all purchased from Sigma. S-adenosylmethionine was purchased from Now Foods (Bloomingdale, Ill.). Insulin and atropine were dissolved in saline. The antioxidants were incorporated into regular rat chow by Research Diets Inc. (New Brunswick, N.J.). Plasma insulin concentration was assayed by ELISA (ALPCO, Windham, N.H.).

Statistical Analysis: Values are presented as means±SE. The data were analyzed by paired or unpaired t-test where appropriate. A one-way ANOVA followed by Tukey's test was employed when the multiple means from different groups were compared. Statistical significance was taken at p<0.05. Linear regression was used.

### Results

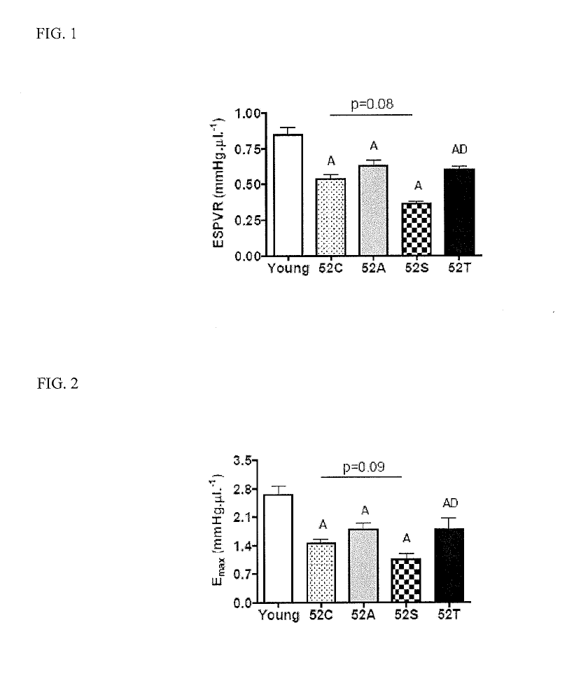

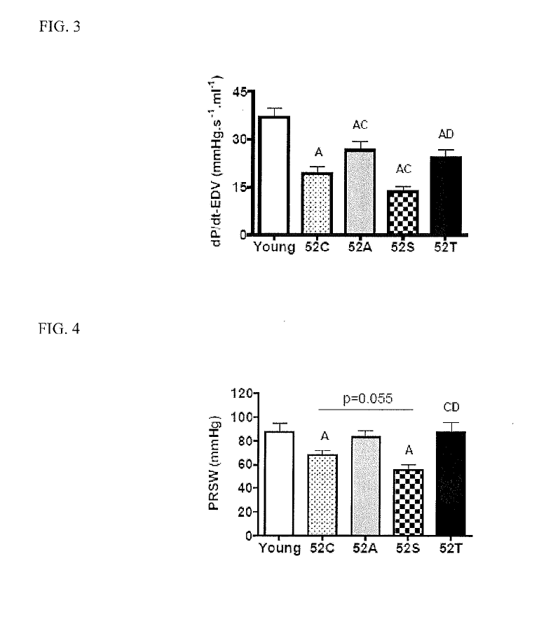

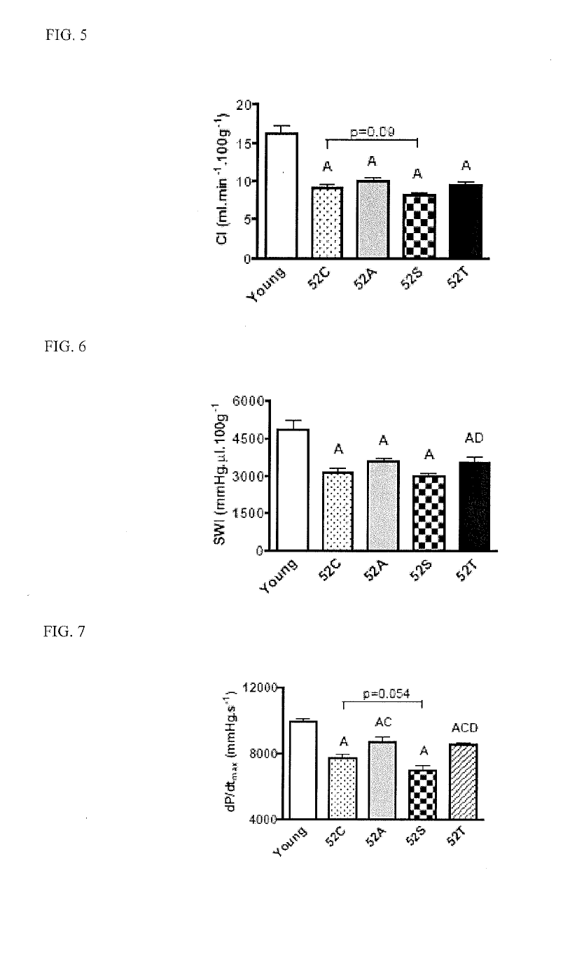

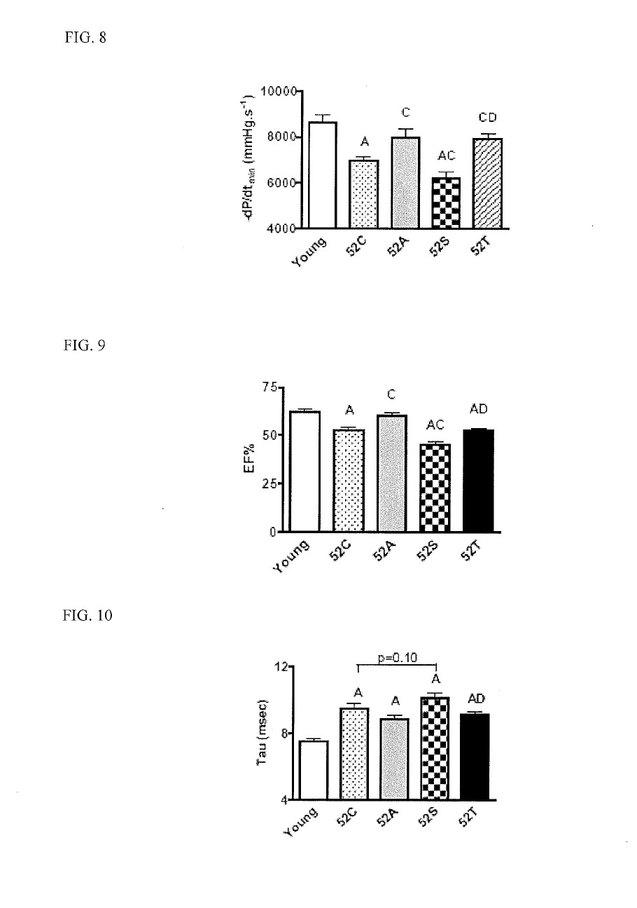

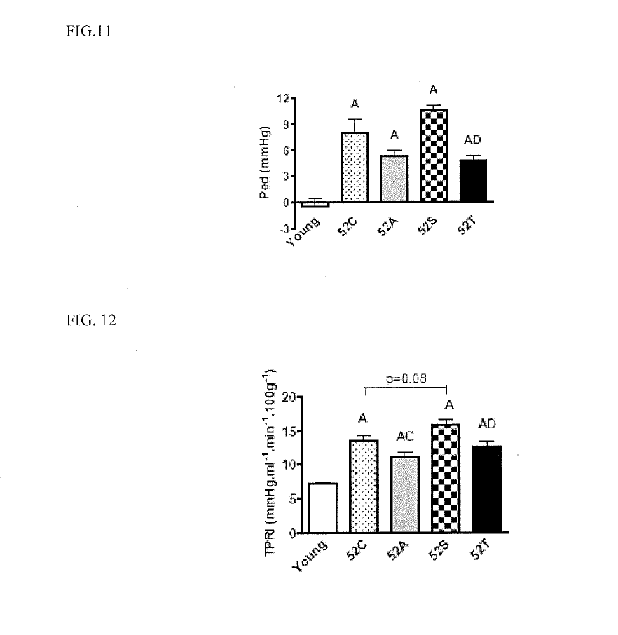

Aging on cardiac function and effects of SAMEC treatment. In 52-week old rats, SAMEC treatment resulted in improved cardiac performance. As shown in FIGS. 1 to 9, rats treated with SAMEC (indicated at column 52A) demonstrated beneficial effects in improving cardiac performance (pressure-volume indexes shown at FIGS. 1-4 and in baseline hemodynamic parameters shown at FIGS. 5-12) at 52 weeks compared to rats given no SAMEC treatment (indicated at column 52C). These beneficial effects included higher: ESPVR (FIG. 1), Eₘₐₓ (FIG. 2), dP/dtₘₐₓ−EDV (FIG. 3), PRSW (FIG. 4), CI (FIG. 5), SWI (FIG. 6), dP/dtₘₐₓ (FIG. 7), dP/dtₘᵢₙ (FIG. 8) and EF % (FIG. 9), compared with rats of the same age having no SAMEC treatment. Beneficial effects also included reduced: Tau (FIG. 10), Ped (FIG. 11) and TPRI (FIG. 12).

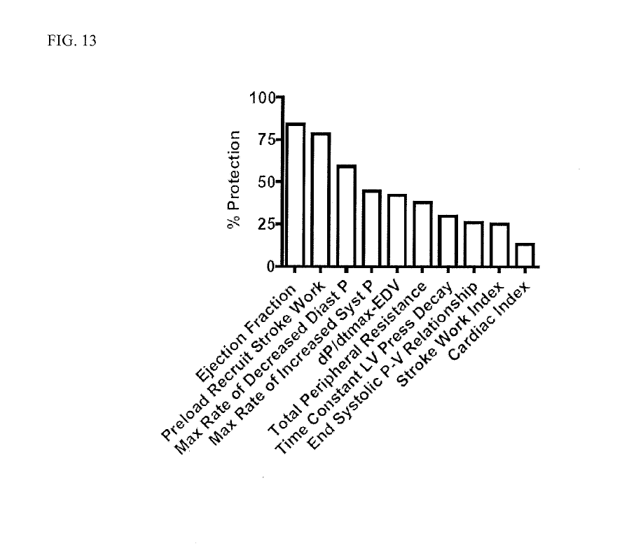

In FIG. 13, the percentage protection provided by SAMEC for impaired cardiac performance associated with aging only is shown in the graph. Percentage protection is calculated as follows: % Protection=[1−(Y−A)/(Y−C)], where Y represents the effect in young 9-week old rats, C represents the effect in 52-week old rats with a control diet, and A represents the effect in 52-week old rats fed a SAMEC diet. SAMEC provided higher protection for EF %, PRSW, dP/dtₘₐₓ, dP/dtₘᵢₙ, dP/dtₘₐₓ−EDV, Total Peripheral Resistance, Time Constant LV Press Decay, End Systolic P-V Relationship, SWI, and CI associated with aging.

Sucrose diet on cardiac function in aging rats and effects of SAMEC treatment. As shown in FIGS. 1 to 9, in 52-week old rats fed sucrose only (indicated at column 52S), there was a tendency for having a lower cardiac performance as compared to their normal diet partners (indicated at column 52C). SAMEC treatment resulted in improved cardiac performance in rats treated with both SAMEC and sucrose (indicated at column 52T) demonstrated beneficial effects in improving cardiac performance at 52 weeks compared to rats given sucrose (indicated at column 52S). These beneficial effects included higher: ESPVR (FIG. 1), E. (FIG. 2), dP/dtₘₐₓ−EDV (FIG. 3), PRSW (FIG. 4), CI (FIG. 5), SWI (FIG. 6), dP/dtₘₐₓ (FIG. 7), dP/dtₘᵢₙ (FIG. 8) and EF % (FIG. 9), compared with rats of the same age having received sucrose only. Beneficial effects also included reduced: Tau (FIG. 10), Ped (FIG. 11) and TPRI (FIG. 12). The improvement of cardiac performance in sucrose rats given SAMEC reached levels similar to their same age partners who were not given sucrose, but were given SAMEC only, namely with respect to the following parameters: ESPVR (FIG. 1), E. (FIG. 2), PRSW (FIG. 4), SWI (FIG. 6), dP/dtₘᵢₙ (FIG. 8), Tau (FIG. 10), and TRPI (FIG. 12).

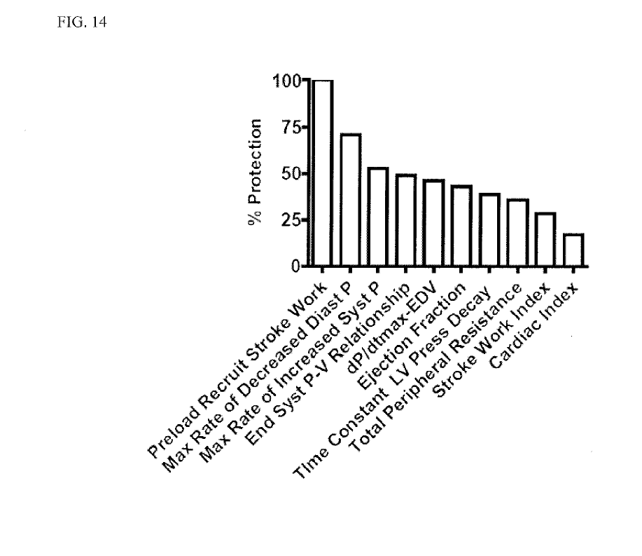

In FIG. 14, the percentage protection provided by SAMEC for impaired cardiac performance associated with aging and a diet high in sucrose is shown in the graph. Percentage protection is calculated as follows: % Protection=[1−(Y−A)/(Y−C)], where Y represents the effect in young 9-week old rats, C represents the effect in 52-week old rats with a control diet, and A represents the effect in 52-week old rats fed a SAMEC diet. SAMEC provided higher protection for PRSW, dP/dtₘᵢₙ, dP/dtₘₐₓ, End Systolic P-V Relationship, dP/dtₘₐₓ−EDV, EF, Time Constant LV Press Decay, Total Peripheral Resistance, SWI, and CI associated with aging and a diet high in sucrose

The pathophysiological characteristics in the murine aging heart are similar to what occur in the elderly human (Dai D F, Rabinovitch P S. Cardiac aging in mice and humans: the role of mitochondrial oxidative stress. Trends Cardiovasc Med 19(7): 213-20, 2009). The present longitudinal study compared cardiac performance in rats during the lifespan of 9, 26 and 52 weeks. In agreement with previous studies, our data showed that both systolic and diastolic cardiac performance declined gradually with aging and became statistically significant at the age of 52 weeks. Evaluated from load-dependent indexes, the decreases in EF %, dP/dtₘₐₓ, dP/dtᵢₙiₙ, and HR were—14, 22, 19, and 16%, respectively. Tau increased by 27%. In addition, load-independent indexes reflecting cardiac intrinsic contractile function, including ESPVR, Emax, dP/dt−EDV and PRSW, also decreased by −36, 45, 47 and 22%, respectively. However, although the inventors observed a significant elevation in end-diastolic pressure which suggested an end-diastolic myocardial stiffness, there was no significant change in EDPVR in 52-week-old rats compared with the young control group.

The purpose of the above description is to illustrate some configurations and uses of the present invention, without implying any limitation. It will be apparent to those skilled in the art that various modifications and variations may be made in the process and product of the invention without departing from the spirit or scope of the invention. All references cited herein are hereby incorporated by reference.

We claim:

1. A method of preventing or treating cardiovascular dysfunction comprising administering a therapeutically effective amount of:
1) one or more of (a) S-adenosylmethionine, a derivative, or a pharmaceutically acceptable salt thereof, and (b) N-acetylcysteine, a derivative, or a pharmaceutically acceptable salt thereof;
2) vitamin E or a derivative or pharmaceutically acceptable salt thereof; and
3) vitamin C or a derivative or pharmaceutically acceptable salt thereof.

2. The method according to claim 1, wherein the cardiovascular dysfunction is chronic.

3. The method according to claim 1, wherein the cardiovascular dysfunction is acute.

4. The method according to claim 1, wherein the cardiovascular dysfunction is related to aging.

5. The method according to claim 1, wherein the cardiovascular dysfunction is related to a high sugar diet.

6. The method according to claim 1, wherein the patient has a high sugar diet.

7. The method according to claim 1, wherein the cardiovascular dysfunction is selected from the group consisting of any of the following: decrease in preload recruitable stroke work (PRSW); decrease in end-systolic pressure-volume relationship (ESPVR); decrease in dP/dtₘₐₓ−end-diastolic volume relationship (dP/dtₘₐₓ−EDV); maximum elastance (Eₘₐₓ); maximal left ventricular systolic pressure (Pes); left ventricular end-diastolic pressure (Ped); the maximal rates of left-ventricular pressure upstroke and fall (dP/dtₘₐₓ and dP/dtₘᵢₙ, respectively); time constant of left ventricular pressure decay (tau); ejection fraction (EF); cardiac output normalized to body weight (cardiac index, CI); stroke work normalized to body weight (SWI); and, total peripheral resistance index (TPRI).

8. The method according to claim 1, wherein the therapeutically effective amount of S-Adenosylmethionine, derivative or pharmaceutically acceptable salt thereof or N-acetylcysteine or a derivative or pharmaceutically acceptable salt thereof, is between 200 and 1600 mg administered orally, daily.

9. The method according to claim 1, wherein the therapeutically effective amount of one or more of S-adenosylmethionine or a derivative or pharmaceutically acceptable salt thereof and N-acetylcysteine or a derivative or pharmaceutically acceptable salt thereof, is 400 mg administered orally, daily.

10. The method according to claim 1, wherein the therapeutically effective amount of vitamin E, a derivative or pharmaceutically acceptable salt thereof, is between 100 and 900 mg administered orally.

11. The method according to claim 1, wherein the therapeutically effective amount of vitamin E, a derivative or pharmaceutically acceptable salt thereof, is 300 mg administered orally.

12. The method according to claim 1, wherein the therapeutically effective amount of vitamin C, a derivative or pharmaceutically acceptable salt thereof, is between 200 mg to 2000 mg administered orally.

13. The method according to claim 1, wherein the therapeutically effective amount of vitamin C, a derivative or pharmaceutically acceptable salt thereof, is 500 mg administered orally.

14. The method according to claim 1, wherein the therapeutically effective amount of one or more of (a) S-adenosylmethionine, a derivative, or a pharmaceutically acceptable salt thereof, and (b) N-acetylcysteine, a derivative, or a pharmaceutically acceptable salt thereof, is 400 mg, the therapeutically effective amount of vitamin E, a derivative or pharmaceutically acceptable salt thereof, is 300 mg, the therapeutically effective amount of vitamin C, a derivative or pharmaceutically acceptable salt thereof is 500 mg, administered orally.

15. A method of claim 1, wherein the administration of (1), (2), (3) occurs at the same time.

16. A method of claim 1, wherein the administration of (1), (2) and (3) occurs on the same day.

17. The use of a therapeutically effective amount of: one or more of (a) S-adenosylmethionine, a derivative, or a pharmaceutically acceptable salt thereof, and (b) N-acetylcysteine, a derivative, or a pharmaceutically acceptable salt thereof; vitamin E or a derivative or pharmaceutically acceptable salt thereof; and vitamin C or a derivative or pharmaceutically acceptable salt thereof; in the manufacture of a medicament for the treatment or prevention of cardiovascular dysfunction.
